(19) United States
(12) Patent Application Publication
(10) Pub. No.: US 2026/0216268 A1
(43) Pub. Date: Jul. 30, 2026

(54) NUTRACEUTICAL MUSHROOM COMPOSITIONS AND METHODS OF TREATMENTS USING SAME

(71) Applicant: Mangoceuticals, Inc., Dallas, TX (US)

(72) Inventors: Natalia Panagiotakopoulos, Golden Beach, FL (US); Bill Panagiotakopoulos, Golden Beach, FL (US); Najla Guthrie, Ontario (CA); Andrew Charrette, Hollywood, FL (US)

(21) Appl. No.: 19/573,414

(22) Filed: Mar. 20, 2026

Related U.S. Application Data
(62) Division of application No. 17/986,502, filed on Nov. 14, 2022.
(60) Provisional application No. 63/278,942, filed on Nov. 12, 2021.

(51) Int. Cl. A61K 36/074; A61K 31/07; A61K 31/165; A61K 31/355; A61K 31/375; A61K 31/51; A61K 31/593; A61K 33/24; A61K 36/068; A61K 36/07; A61K 36/185; A61K 36/19; A61K 36/81; A61K 36/87

(52) U.S. Cl. CPC A61K 36/074; A61K 31/07; A61K 31/165; A61K 31/355; A61K 31/375; A61K 31/51; A61K 31/593; A61K 33/24; A61K 36/068; A61K 36/07; A61K 36/185; A61K 36/19; A61K 36/81; A61K 36/87

(57) ABSTRACT

Disclosed herein are compositions of nutraceuticals for use as dietary supplements and their use for supporting immunity, enhancing mood, improving well-being, improving cognitive function, providing adaptogenic support, providing antioxidant support, and suppressing appetite.

### CROSS-REFERENCE TO RELATED APPLICATIONS

This Application claims the benefit of U.S. Provisional Application 63/278,942 filed on Nov. 12, 2021, the contents of which are incorporated herein in its entirety.

### SUMMARY

Embodiments of the present disclosure relate to compositions of nutraceuticals for use as dietary supplements and their use for supporting immunity, enhancing mood, improving well-being, improving cognitive function, providing adaptogenic support, providing antioxidant support, and suppressing appetite.

These and other feature, aspects, and advantages of the present embodiments will become understood with reference to the following description, appended claims, and accompanying figures.

### DETAILED DESCRIPTION

Some embodiments provide a composition comprising an amount of at least one fungal compound formulated as a fungal composition. The at least one fungal compound may be Cordyceps sinensis or an extract thereof, Ganoderma lucidum (Reishi mushroom) or an extract thereof, Hericium erinaceus (Lion's mane mushroom) or an extract thereof, Pleurotus eryngii (King trumpet mushroom) or an extract thereof, Inonotus obliquus (Chaga mushroom) or an extract thereof, Grifola frondosa (Maitake mushroom), or any combination thereof. In some embodiments, a fungal composition can be formulated to have varying amounts of at least one fungal compound.

In certain embodiments, a fungal composition, as described herein, comprising an amount of at least one fungal compound, as described herein, may comprise about 10 mg to about 1000 mg of the at least one fungal compounds. For example, the amount of the at least one fungal compound in the composition may be about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

In some embodiments, a fungal composition, as described herein, can comprise an amount of a first fungal compound and an amount of a second fungal compound. In some embodiments, the first fungal compound is Cordyceps sinensis or an extract thereof and the second fungal compound is Ganoderma lucidum or an extract thereof. In certain embodiments, the first fungal compound is Cordyceps sinensis or an extract thereof and the second fungal compound is Hericium erinaceus or an extract thereof. In some embodiments, the first fungal compound is Cordyceps sinensis or an extract thereof and the second fungal compound is Pleurotus eryngii or an extract thereof. In certain embodiments, the first fungal compound is Cordyceps sinensis or an extract thereof and the second fungal compound is Inonotus obliquus or an extract thereof. In some embodiments, the first fungal compound is Cordyceps sinensis or an extract thereof and the second fungal compound is Grifola frondosa or an extract thereof. In certain embodiments, the first fungal compound is Ganoderma lucidum or an extract thereof and the second fungal compound is Hericium erinaceus or an extract thereof. In some embodiments, the first fungal compound is Ganoderma lucidum or an extract thereof and the second fungal compound is Pleurotus eryngii or an extract thereof. In certain embodiments, the first fungal compound is Ganoderma lucidum or an extract thereof and the second fungal compound is Inonotus obliquus or an extract thereof. In other embodiments, the first fungal compound is Ganoderma lucidum or an extract thereof and the second fungal compound is Grifola frondosa or an extract thereof. In some embodiments, the first fungal compound is Hericium erinaceus or an extract thereof and the second fungal compound is Pleurotus eryngii or an extract thereof. In certain embodiments, the first fungal compound is Hericium erinaceus or an extract thereof and the second fungal compound is Inonotus obliquus or an extract thereof. In some embodiments, the first fungal compound is Hericium erinaceus or an extract thereof and the second fungal compound is Grifola frondosa or an extract thereof. In certain embodiments, the first fungal compound is Pleurotus eryngii or an extract thereof and the second fungal compound is Inonotus obliquus or an extract thereof. In other embodiments, the first fungal compound is Pleurotus eryngii or an extract thereof and the second fungal compound is Grifola frondosa or an extract thereof. In some embodiments, the first fungal compound is Inonotus obliquus or an extract thereof and the second fungal compound is Grifola frondosa or an extract thereof.

In certain embodiments, a fungal composition comprising an amount of two or more fungal compound may be formulated to provide the two or more fungal compounds in a synergistic ratio. The synergistic ratio, as described herein, may elicit a synergistic response in a subject receiving the fungal composition. The synergistic response, as described herein, may occur with respect to improvement in immunity, enhancement of mood, improvement in general well-being metrics, improvement in cognitive function, greater levels of adaptogenic support, increased levels of antioxidants, or any combination thereof. In certain embodiments, a synergistic response can include maintaining or supporting immune function, maintaining or supporting a healthy mood, maintaining cognitive function, maintaining adaptogenic support, and/or maintaining antioxidant activity, or any combination thereof.

In some embodiments, a synergistic ratio of an amount of a first fungal compound to an amount of a second fungal compound can be about I:1. In certain embodiments, a synergistic ratio of an amount of a first fungal compound to an amount of a second fungal compound can be within the range of about 10: I to about I:10. In this regard, a synergistic ratio of an amount of a first fungal compound to an amount of a second fungal compound may be about 2:1, about 3:1, about 4:1, about 5:1, about 6:1, about 7:1, about 8:1, about 9:1, about 10:1, about 1:2, about 1:3, about 1:4, about 1:5, about 1:6, about 1:7, about 1:8, about 1:9, or about 1:10, or any ratio therebetween.

In certain embodiments, a fungal composition, as described herein, can comprise an amount of three or more fungal compounds, four or more fungal compounds, five or more fungal compounds, or six fungal compounds, as described herein, provided in a synergistic ratio. The relative amount of any fungal compound provided in a combination of three or more fungal compounds is not particularly limited. By way of example, when a fungal composition comprises three fungal compounds, the synergistic ratio of the three fungal compounds can be understood as comprising a “part” of three selected from the group of Cordyceps sinensis or an extract thereof, Ganoderma lucidum or an extract thereof, Hericium erinaceus or an extract thereof, Pleurotus eryngii or an extract thereof, Inonotus obliquus or an extract thereof, and Grifolafrondosa or an extract thereof. For example, a fungal composition may comprise a synergistic ratio of 4: I:5, Cordyceps sinensis to Ganoderma lucidum to Hericium erinaceus, corresponding to 4 parts Cordyceps sinensis, I part Ganoderma lucidum, and 5 parts Hericium erinaceus. Preferably, the individual amount of a fungal compound may be as low as I part, may be as high as 10 parts, or may be any value therebetween, when a fungal compound is present in a fungal composition comprising at least three fungal compounds.

In some embodiments, a fungal composition, as described herein, may further comprise an amount of one or more of a grape seed extract, a birch bark extract, Andrographis paniculate (King of Bitters) or an extract thereof, Capsicum annum or an extract thereof, capsaicin, chromium picolinate, minerals, vitamins (A, B, C, D, or E), or any combination thereof.

As described herein, a grape seed extract may include, but are not limited to one or more proanthocyanidins, which may be synthesized or extracted from grape seed. When provided in a fungal composition, as described herein, the amount of a grape seed extract may be about IO mg to about 1000 mg. For example, the amount of a grape seed extract in the composition may be about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, a birch bark extract can include, but are not limited to, one or more triterpenes. Triterpenes include, but are not limited to betulin, betulinic acid, lupeol, and betulin caffeate. When provided in a fungal composition, as described herein, the amount of a birch bark extract may be about 10 mg to about 1000 mg. For example, the amount of a birch bark extract in the composition may be about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, extracts of Andrographis paniculate may include, but are not limited to one or more of flavonoids and diterpenoids. When provided in a fungal composition, as described herein, the amount of Andrographis paniculate or an extract thereof may be about 10 mg to about 1000 mg. For example, the amount of Andrographis paniculate or an extract thereof in the composition may be about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, I 000 mg, or any range or amount in between any two of the preceding values and any other ranges or amounts disclosed herein.

When provided in a fungal composition, as described herein, the amount of Capsicum annum or an extract thereof may be about 0.01 μg to about 1000 mg. For example, the amount of Capsicum annum or an extract thereof in the composition may be about 0.01 μg, 0.1 μg, I μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

When provided in a fungal composition, as described herein, the amount of capsaicin may be about 0.01 μg to about 1000 mg. For example, the amount of capsaicin in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

When provided in a fungal composition, as described herein, the amount of chromium picolinate may be about 0.01 μg to about 1000 mg. For example, the amount of chromium picolinate in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, minerals may include, but are not limited to one or more of magnesium, potassium, selenium, and zinc. When provided in a fungal composition, as described herein, the amount of a mineral may be about 0.01 μg to about 1000 mg. For example, the amount of a mineral in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, vitamin A refers to compounds that may include, but are not limited to one or more of retinal, retinal, retinoic acid, and carotenoid compounds and derivatives thereof. When provided in a fungal composition, as described herein, the amount of vitamin A may be about 0.01 μg to about 1000 mg. For example, the amount of vitamin A in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, vitamin B refers to compounds that may include, but are not limited to one or more of thiamine (vitamin B1), riboflavin (vitamin B2), niacin (vitamin B3), pantothenic acid (vitamin B5), pyridoxine, pyridoxamine, or pyridoxal (vitamin B6), biotin (vitamin B7) or pharmaceutically acceptable salts thereof, folic acid (vitamin B9) or pharmaceutically acceptable salts thereof, and cobalamin (vitamin B12). When provided in a fungal composition, as described herein, the amount of vitamin B may be about 0.01 μg to about 1000 mg. For example, the amount of vitamin B in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, vitamin C refers to compounds that may include, but are not limited to one or more of ascorbic acid, ascorbate, or pharmaceutical salts thereof. When provided in a fungal composition, as described herein, the amount of vitamin C may be about 0.01 μg to about 1000 mg. For example, the amount of vitamin C in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, vitamin D refers to compounds that may include, but are not limited to one or more of a mixture of ergocalciferol and lumisterol (vitamin D1), ergocalciferol (vitamin D 2), calciferol (vitamin D 3), 22-dihydroergocalciferol (vitamin D 4), and sitocalciferol (vitamin D5). When provided in a fungal composition, as described herein, the amount of vitamin D may be about 0.01 μg to about 1000 mg. For example, the amount of vitamin D in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, or any range or amount in between any two of the preceding values and any other ranges or amounts disclosed herein.

As described herein, vitamin E refers to compounds that may include, but are not limited to one or more of tocopherols and tocotrienols. When provided in a fungal composition, as described herein, the amount of vitamin E may be about 0.01 μg to about 1000 mg. For example, the amount of vitamin E in the composition may be about 0.01 μg, 0.1 μg, 1 μg, 10 μg, 50 μg, 100 μg, 200 μg, 300 μg, 400 μg, 500 μg, 600 μg, 700 μg, 800 μg, 900 μg, 1 mg, 2.5 mg, 5 mg, 7.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, s 825 mg, 850 mg, 875 mg, 900 mg, 925 mg, 950 mg, 975 mg, 1000 mg, or any range or amount in between any two of the preceding values and any other ranges or amounts disclosed herein.

As described herein, Cordyceps sinensis or extracts thereof may contain compounds including, but not limited to bioactive polysaccharides, modified nucleosides, cyclosporine-like metabolites, adenosine, adenosine monophosphate, adenosine diphosphate, adenosine triphosphate, uridine-5′-monophosphate adenosine-5′-monophosphate, guanosine-5′-monophosphate, exopolysaccharide fraction, cordycepic acid (D-mannitol), and combinations thereof. When contained in a fungal composition, these compounds may support an improvement in energy, stamina, antioxidant activity, anti-aging properties, heart health, aerobic metabolic activity, adaptogenic properties, stress response, immune function, anti-inflammatory properties, prevention of tissue damage, anticonvulsant activity, fatty acid metabolism, iron absorption, repair of the gastrointestinal tract, scavenging free radicals, treatment of liver fibrosis, or any combination thereof.

As described herein, Ganoderma lucidum or extracts thereof may contain triterpene compounds, including, but not limited to lanostane-tetracyclic triterpenes. When contained in a fungal composition, these compounds may support an improvement in, or support healthy levels of, immune function, antioxidant activity, cardiovascular function, adaptogenic properties, cognitive function, reduction of stress levels, energy levels, anti-inflammatory properties, antioxidant activity, antimicrobial activity, reduction of blood lipid levels, neurotrophic activity, treatment of neurodegenerative diseases, such as Alzheimer's Disease and Parkinson's Disease, or any combination thereof.

As described herein, Hericium erinaceus or extracts thereof may contain compounds, including, but not limited to erinacines and hericenones. When contained in a fungal composition, these compounds may support an improvement in, or support healthy levels of, adaptogenic properties, feelings of balanced mind and body, antioxidant activity, cognitive function, memory, nerve growth factor synthesis in nerve cells, or any combination thereof.

As described herein, Pleurotus eryngii or extracts thereof may contain compounds, including, but not limited to L-ergothioneine, selenium, and polysaccharides. When contained in a fungal composition, these compounds may support an improvement in, or support healthy levels of, antioxidant activity, reduction of free radicals, anti-inflammatory properties, maintenance of healthy blood lipid levels and body weight, immune function, healthy bone mineralization, or any combination thereof.

As described herein, Inonotus obliquus or extracts thereof may contain compounds, including, but not limited to triterpenoids, betulin, inotodiol, trametenolic acid, betulinic acid, B-complex vitamins, vitamin D, potassium, magnesium, zinc, and selenium. When contained in a fungal composition, these compounds may support an improvement in, or maintain healthy levels of, reduction of oxidative stress, immune function, or any combination thereof.

As described herein, Grifola frondosa or extracts thereof may contain compounds, including, but not limited to 1,3 or 1,6 beta-glucan compounds. When contained in a fungal composition, these compounds may support an improvement in, or support healthy levels of, immune function, reduction of severe symptoms of the common cold, or any combination thereof.

Certain embodiments of a fungal composition, as described herein, can be formulated as a nutraceutical for use as a dietary supplement for suppressing appetite, for enhancing mood and well-being, for supporting immunity, for improving cognitive function, for providing adaptogenic support, for providing antioxidant support, or any combination thereof. The fungal composition may comprise at least one fungal compound selected from the group consisting of Cordyceps sinensis or an extract thereof, Ganoderma lucidum (Reishi mushroom) or an extract thereof, Hericium erinaceus (Lion's mane mushroom) or an extract thereof, Pleurotus eryngii (King trumpet mushroom) or an extract thereof, Inonotus obliquus (Chaga mushroom) or an extract thereof, Grifola frondosa (Maitake mushroom), or any combination thereof. The fungal composition may further comprise one or more of a grape seed extract, a birch bark extract, Andrographis paniculate (King of Bitters) or an extract thereof, Capsicum annum or an extract thereof, capsaicin, chromium picolinate, minerals, vitamins (A, B, C, D, or E), or any combination thereof.

An embodiment of a fungal composition of the disclosure can comprise about 50 micrograms of Vitamin D 2, about 15 mg of zinc, about 300 mg of Andrographis paniculata leaf extract, about 200 mg of Grifola frondose extract, and about 100 mg of a blend of Ganoderma lucidum, Cordyceps sinensis, Pleurotus eryngii, and Hericium erinaceus.

An embodiment of a fungal composition of the disclosure can comprise about 500 mg of Inonqus obliquus, about 250 mg of grape seed extract, about 150 of birch bark extract, and about 50 mg of a blend of Ganoderma lucidum, Cordyceps sinensis, Pleurotus eryngii, and Hericium erinaceus.

An embodiment of a fungal composition of the disclosure can comprise about 900 mcg Vitamin A, about 90 mg of Vitamin C, about 20 mcg of Vitamin D 2, about 15 mg of Vitamin E, about 1.2 mg of Vitamin B1, about 1.3 mg of Vitamin B2, about 16 mg of Vitamin B6, about 400 mcg of Folate, about 40 mcg of chromium picolinate, and about 50 mg of Capsicum annum L. extract.

Some embodiments relate to fungal compositions comprising one or more compositions disclosed herein, with at least one pharmaceutically acceptable excipient, or vehicle. For example, pharmaceutically acceptable vehicles can include carriers, excipients, diluents, and the like, as well as combinations or mixtures thereof. As used herein, an “excipient” refers to a substance that is added to a composition for the purpose of long-term stabilization, bulk, consistency, binding ability, lubrication, disintegrating ability, drug absorption, enhanced solubility etc. A “diluent” is a type of excipient.

As used herein, a “carrier” refers to a compound that interacts with and enhances the properties of active ingredients or which facilitates the incorporation of a compound into cells or tissues of a subject.

As used herein, a “diluents” refers to a constituent that acts as filler in a formulation to increase weight and improve content uniformity. The diluent may be necessary or desirable in a formulation.

The fungal compositions disclosed herein can take the form of solutions, suspensions, emulsion, tablets, pills, pellets, capsules, capsules containing liquids, powders, sustained-release formulations, suppositories, emulsions, aerosols, sprays, chewable dosage forms, gelatin dosage forms, liquid dosage forms, or any other form suitable for use.

In some embodiments, the compositions disclosed herein are administered through various delivery modes including, but not limited to, orally, intravenously, or subcutaneously. Other modes of administration of the compositions include, but are not limited to, intradermal, intramuscular, or intraperitoneal. The compositions described herein can be administered to human subjects. In some embodiments, the compositions are mixed with other active ingredients, or carriers, diluents, excipients, or combinations thereof.

The compositions intended for oral administration can be prepared according to any method known in the art for manufacturing the compositions, and such compositions may also contain at least one of the agents, namely, sweeteners, flavoring agents, coloring agents, and preservatives.

The compositions disclosed herein can be assayed in vitro and in vivo for the desired therapeutic, nutritional, or prophylactic activity prior to use in human subjects. For example, in vivo assays can be used to determine whether administration of a specific compound, as described herein, or a combination of compositions disclosed herein is preferred for treating, preventing, ameliorating, or preventing age-related somatic diseases such as fatigue, depression, chronic pain, and the like. Composition can also be used to treat and/or prevent bacterial or viral infections. The compositions disclosed herein can also be shown to be effective and safe in animal models or animal subjects.

As used herein, the terms “synergy”, “synergistic”, “synergism” and the like are used herein to generally refer to the efficacy of a composition being at least equal to the sum of the efficacy of the individual components in the composition administered independently. “Synergy”, “synergistic”, “synergism” and the like may also refer to the efficacy of a composition being greater than to the sum of the efficacy of the individual components in the composition administered independently. The scopes and meanings of which will be clear to the skilled artisan based upon the context in which these terms are used.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

1.-7. (canceled)

8. A method of improving cognitive function in a subject, the method comprising:
administering to the subject a fungal composition, wherein the fungal composition comprises a fungal compound and a compound,
wherein the fungal compound is selected from the group consisting of Cordyceps sinensis or an extract thereof, Ganoderma lucidum or an extract thereof, Hericium erinaceus or an extract thereof, Pleurotus eryngii or an extract thereof, Inonotus obliquus or an extract thereof, Grifola frondosa, and any combination thereof; and
wherein the compound is selected from the group consisting of a grape seed extract, a birch bark extract, Andrographis paniculate or an extract thereof, Capsicum annum or an extract thereof, capsaicin, chromium picolinate, a mineral, vitamin A, vitamin B, vitamin C, vitamin D, vitamin E, and any combination thereof.

9. The method of claim 8, wherein the fungal compound is Cordyceps sinensis or an extract thereof.

10. The method of claim 8, wherein the fungal compound is Hericium erinaceus or an extract thereof.

11. The method of claim 8, wherein the compound is a grape seed extract.

12. The method of claim 8, wherein the compound is a birch bark extract.

13. The method of claim 8, wherein the fungal composition is formulated for delivery to the subject through a route selected from the group consisting of oral, intravenous, subcutaneously, intradermal, intramuscular, and intraperitoneal.

14. A method of improving immune function in a subject, the method comprising:
administering to the subject a fungal composition, wherein the fungal composition comprises a fungal compound and a compound,
wherein the fungal compound is selected from the group consisting of Cordyceps sinensis or an extract thereof, Ganoderma lucidum or an extract thereof, Hericium erinaceus or an extract thereof, Pleurotus eryngii or an extract thereof, Inonotus obliquus or an extract thereof, Grifola frondosa, and any combination thereof; and
wherein the compound is selected from the group consisting of a grape seed extract, a birch bark extract, Andrographis paniculate or an extract thereof, Capsicum annum or an extract thereof, capsaicin, chromium picolinate, a mineral, vitamin A, vitamin B, vitamin C, vitamin D, vitamin E, and any combination thereof.

15. The method of claim 14, wherein the fungal compound is Cordyceps sinensis or an extract thereof.

16. The method of claim 14, wherein the fungal compound is Hericium erinaceus or an extract thereof.

17. The method of claim 14, wherein the compound is a grape seed extract.

18. The method of claim 14, wherein the compound is a birch bark extract.

19. The method of claim 14, wherein the fungal composition is formulated for delivery to the subject through a route selected from the group consisting of oral, intravenous, subcutaneously, intradermal, intramuscular, and intraperitoneal.
